(19) United States
(12) Patent Application Publication
(10) Pub. No.: US 2010/0075410 A1
(43) Pub. Date: Mar. 25, 2010

(54) Reusable Tissue Biopsy Kit with Padded Cassette

(76) Inventors: Virendra Desai, Jacksonville, FL (US); Jignasa Desai, Jacksonville, FL (US)

Correspondence Address: Matthew P. Davies, 13870 Devan Lee Drive North, Jacksonville, FL, 32226, US

(21) Appl. No.: 12/234,626

(22) Filed: Sep. 20, 2008

(51) Int. Cl. C12M 1/22; B01L 3/00

(52) U.S. Cl. 435/299.1; 422/102

(57) ABSTRACT

An improved biopsy kit for the collection, storage, transportation, and processing of biopsy tissue samples is disclosed. The disclosed biopsy kit consists of a typical specimen container and an improved tissue cassette with adhesively attached polyvinyl alcohol foam pads on both the base and lid portions. An excised tissue sample is sandwiched between the pads upon cassette closure, simultaneously isolating and securing the sample in its original orientation. Superior wicking, absorbency, and moisture retention rate of these polyvinyl alcohol pads allows maximum tissue fixation as well as maintains sample integrity. Once the tissue is placed by the physician at the patient's side, it remains there until the tissue is ready to be processed. This eliminates transfer errors as well as tissue loss.

### CROSS-REFERENCES

None

### GOVERNMENT RIGHTS

None

### OTHER PUBLICATIONS

D J Farrell, P J Thompson and A R Morley, Tissue artefacts caused by sponges, J. Clin. Pathol. 1992;45;923-924
George S. Davidson and Karel G. Terbrugge, Histologic Long-term Follow-up after Embolization with Polyvinyl Alcohol Particles, AJNR 16:843-846, April 1995 0195-6108/95/1604-0843.
Ye C, Chen H, Zou H, Pan Z, Peng Y., Study of the polyvinyl alcohol-collagen blend as wound dressing, Guangzhou Red Cross Hospital, The Affiliate of the Medical School of Jinan University, June 2008;25(3), 604-6, Guangzhou, 510220, China.
Niemann T. H.⁽¹⁾; Tranovich J. G.⁽¹⁾; De Young B. R.⁽¹⁾, Biopsy bag artifact, Department of Pathology, Ohio State University and the Arthur G. James Cancer Hospital and Research Institute, Columbus, Ohio, ETATS-UNIS.

### BACKGROUND OF INVENTION

### Field of Invention

This invention relates generally to a reusable tissue biopsy kit.

### Discussion

Biopsy kits are well known in the prior art for the collection, transportation, processing, and storage of biopsy tissue samples. Traditional means of sample storage use vials/containers with fixative solution and/or plastic tissue cassettes. These cassettes are hinged so as to include the container attached to a lid that snaps shut after the sample is placed inside. Both the lid and the container contain perforations to allow solvent to flow through the cassette to keep the sample moist.

Typical biopsy tissue samples transfer from physician to pathologist in a manner similar to that which follows. Biopsy samples may be taken from multiple sites of the same organ. Each biopsy sample must be placed in a separate fixative container (commonly a 10% formalin solution). Therefore, the physician is required to label each fixative container with detailed patient information as well as specific site information from where the biopsy is being removed. A tissue sample (or samples) is excised from a patient using any number of devices including needles, fiberoptics, etc. Some of these devices incorporate internal temporary storage cells to hold a sample (or samples) through the completion of the procedure. Many devices, however, do not incorporate such temporary storage cells. In either case the excised tissue sample is transferred with a pair of forceps to a temporary storage vessel containing fixative (or preservative). The tissue sample is then transferred again with a pair of forceps to a piece of filter paper or a small sponge and then transferred into a cassette. The cassette is closed, then placed in either a container, or a bag containing fixative (commonly formalin solution, a known lachrymator and a possible carcinogen) for transfer to a pathology lab for processing. At this stage the tissue sample is freely floating in the fixative solution. Upon arriving at the pathology lab, a pathologist must remove the cassette (or tissue sample in the case of a fixative container) from the transportation or fixative container, exposing the pathologist to varying amounts of formalin. The sample must then be dehydrated in preparation for processing (microtome, etc.). In some cases the cassettes may be placed directly into an automated chemical processing machine. In others the sample must be transferred from one cassette to another using a pair of forceps.

Several drawbacks to this process exist. The first of which is the lack of sample orientation during transport. As the tissue sample is not secured in place it does not retain the original orientation within the cassette as placed by the physician. Additionally because the tissue sample is freely floating in the fixative solution, its gross appearance may change. A common problem is the bending or curving of the tissue sample into a crescent shape. When this occurs it takes more time for the pathologist to interpret the resultant slide and can potentially compromise the diagnosis.

A second drawback concerns the tissue artifacts that arise through the use of foam sponges and nylon in tissue cassettes. In the aforementioned drawback the tissue sample is not fixed and may move about within the cassette. When these tissues are placed on sponges the tissue is distorted due to the microstructure of the sponge which is composed of a mesh of scimitar-shaped ridge spikes. Thus sponges are responsible for a range of artifacts depending on tissue size, shape, and degree of fixation. The use of nylon biopsy bags and nylon mesh in cassettes can cause artifacts ranging from a tic-tac-toe pattern, to elongated oval spaces, to a serrated contour at the periphery of the tissue. In conclusion, these artifacts can interfere with biopsy interpretation and potentially compromise the diagnosis.

A third drawback concerns the size of the perforations in the cassette. As such the physical size of the excised tissue can be no smaller than the dimensions of the perforations in the cassette. This could potentially limit the type and number of tests performed on a biopsy sample.

A fourth drawback concerns the unnecessary exposure of personnel (physicians, pathologists, technicians, etc.) to formalin vapors. In the case of vials/fixative containers the exposure is prolonged because the tissue samples are freely floating in the fixative solution taking longer to search and transfer to the processing cassettes. With the use of cassettes stored in formalin solution, the cassettes pull solution out of the storage container when the cassettes are transferred. This solution then evaporates into the atmosphere. The aforementioned process must be repeated for each biopsy site. As numerous specimens/cassettes are processed daily the build up of formalin vapors can be quite large causing a substantial health hazard. Additionally after a cassette is removed from its formalin storage container, possible error arises as the sample may dry out if left unattended for even relatively short periods of time.

A fifth drawback concerns the fact that upon submersion of the tissue sample into the formalin container, the formalin solution becomes contaminated and thereby non reusable. Typically, with the use of vials/containers containing fixative solution at least 20 ml of formalin solution is used per biopsy site to preserve the specimen. Additionally when tissue cassettes are used, due to their size the volume of formalin required to preserve the sample can be even greater. As such large volumes of solution are wasted after only one use. This creates disposal issues and environmental hazards, as well as both the monetary and chronological costs involved in preventing the pathologist from analyzing the sample.

A sixth drawback comes about through the repeated use of forceps to transfer the sample to various containers for processing. Not only does this jeopardize the integrity of the sample but additionally increases the probability of cross-contamination through the introduction of foreign material. Additionally the use of forceps at multiple stages of sample processing increases the likelihood of transfer errors. Typically pathologists receive multiple tissue samples from numerous biopsy patients during the course of a day. As such the of confusing a set of samples from different patients or from within the same patient, increases exponentially.

A final drawback to this process involves the time it takes a pathologist to properly complete sample transfer from fixative containers to corresponding processing cassettes. The more time the pathologist is involved with transfer the less time the pathologist has for diagnosis and the more costly the entire process becomes.

One solution is to use a non-perforated cassette made of porous material that is prefilled with fixative solution to aid in sample transfer. Another such solution employs the use of a standard sponge placed into the cassette to keep the sample moist. Yet another solution combines the cassette and storage fixative container into one device. These devices, however, add to the overall complexity and manufacturing costs. Several solutions to these issues exist, however none claim to simultaneously solve all of these problems. Therefore it is the object of this invention to solve one or more of these problems.

### SUMMARY OF INVENTION

In accordance with the teachings of this invention as embodied and described herein, an improved tissue biopsy kit with padded tissue cassette is disclosed. The present invention presents a tissue biopsy kit that includes an improved tissue cassette with attached lid, a sealable container to house the fixative solution and improved tissue cassette, and a lid to seal the container for processing. Using the principles of the invention a biopsy kit may be implemented as a device that provides for the collecting, storage, transportation, and processing of biopsy tissue samples, as well as a device that eliminates artifacts, cross-contamination, transfer error, extensive processing time, and exposure of the user to potentially harmful chemicals and samples.

In one embodiment a tissue cassette with perforations on the top and bottom portions incorporates a polyvinyl alcohol (PVA) foam pad secured to the base portion with a water soluble polyvinyl alcohol based adhesive. A second polyvinyl alcohol foam pad is adhesively secured to the lid portion of the cassette.

A number of benefits can be derived through the use of a tissue cassette incorporating a polyvinyl alcohol foam pad. First, the tissue sample may be properly secured (by the physician) in its original orientation upon closing or mating the cassettes lid portion with its base portion. As the sample is sandwiched between two polyvinyl alcohol pads it is completely isolated from the user and/or other samples that may be stored in the same container. As the polyvinyl alcohol sandwich is placed in the aqueous fixative solution the water soluble adhesive dissolves thereby allowing the sandwich to separate from the cassette without compromising the integrity of the sample. This stands to eliminate sample handling and processing errors as well as protects the user from potentially harmful diseases (such as HIV) and allows the fixative solution to be reused protecting the environment. The polyvinyl alcohol foam pads, and tissue cassette can be reused for tissue processing.

Secondly, the polyvinyl alcohol foam pad is completely biocompatible, biodegradable and lint free. As such the polyvinyl alcohol foam pad will not react with tissue samples to create the negative artifacts commonly associated with other sponge materials (cellulose, polyurethane, nylon, etc. . . . ). This manifestation can be attributed to the smooth, porous, and nonabrasive properties of the polymer.

Lastly, it can be shown that due to its hydrophilic properties the polyvinyl alcohol foam pad has superior wicking, absorption, and moisture retention rates to preserve the sample for longer periods of time. Additionally the polyvinyl alcohol foam pad is opaque to visible and UV light to further guard against sample degradation.

By utilizing the current biopsy kit the potential errors, dangers of biopsy tissue contamination and processing, and loss of user time associated with the traditional biopsy kits are avoided. Thus the tissue sample is securely sandwiched within the tissue cassette without cause for mal alignment leading to artifacts, giving superior processing results.

### BRIEF DESCRIPTION OF DRAWINGS

FIG. 1 is a top perspective view of the tissue cassette in the open position showing the polyvinyl alcohol pad secured in the interior.

FIG. 2 is a bottom perspective view of the tissue cassette in the open position showing the solvent access perforations.

FIG. 3 is a top perspective view of the tissue cassette in the closed position.

FIG. 4 is a top perspective view of the cassette storage container, tissue cassette, and the container lid.

FIG. 5 is a graph that compares the wicking rate of the current invention to that which is known in the art.

FIG. 6 is a graph that illustrates the comparison of absorbency between polyvinyl alcohol and other materials known in the art.

FIG. 7 is a graph that illustrates the moisture retention rate of the polyvinyl alcohol pad.

### DETAILED DESCRIPTION OF THE INVENTION

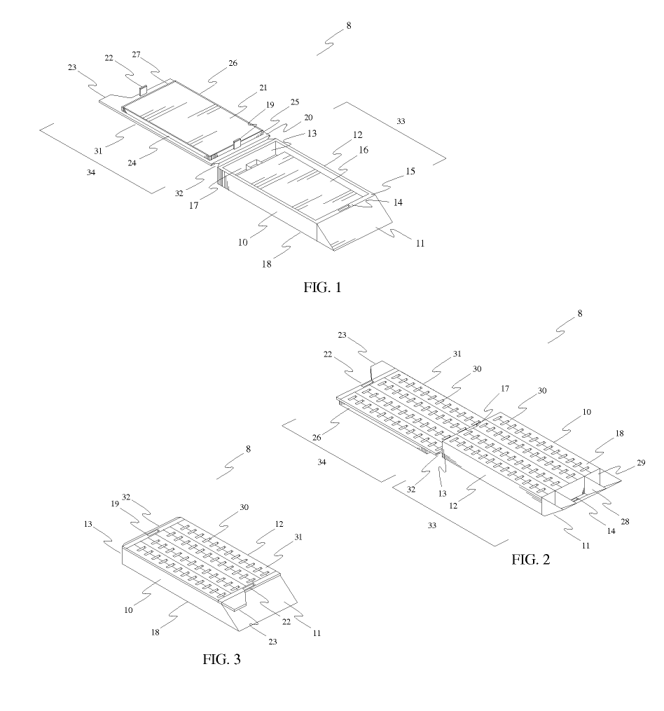

Referring now to the drawings, and more particularly FIGS. 1-3, the present invention includes a tissue cassette 8 in the open position with a base portion 33 attached to a lid portion 34 by a living hinge 32. The base portion 33 includes opposed side walls 10 and 12, a back wall 13, a support wall 28, and front wall 29, depending perpendicularly from a bottom wall 18. Back wall 13, opposing side walls 10 and 12, and the front wall 29, are joined at their respective ends and have portions defining a compartment. The base portions 33 having portions defining a compartment includes a polyvinyl alcohol (PVA) foam pad 16 within said compartment and is adhesively attached to the bottom wall 18. The aforementioned adhesive securing the polyvinyl alcohol foam pad 16 to the bottom wall is polyvinyl alcohol based. Further the polyvinyl alcohol based adhesive is water soluble for the purpose of dissolving upon insertion into the fixative solution, thus allowing the sandwich to be removed from the cassette 8 upon processing. As shown in FIG. 2 the front wall 29 is joined perpendicularly to the support walls 28 which are attached perpendicularly to a slanted front wall 11. The front face of the slanted front wall 11 may contain pertinent information, via directly writing on the surface, or affixing an adhesive label that may contain words, numbers, bar codes, RFID tags or the like. The top surface of the opposing side walls 10 and 12, back wall 13, and front wall 29 form a lip 15 for releasably mating the lid portion 34 with the base portion 33. The lip portion 15 of the front wall 29 may contain portions defining a front transverse slot 14, which may include a receiving portion for the purpose of releasably engaging the front locking tab 22. The bottom wall 18 may include a plurality of perforations 30 and a back transverse slot 17. Back transverse slot 17 may include a receiving portion for the purpose of releasably engaging the back locking tab 19.

The lid portion 34 includes a second front wall 27, a second set of opposed walls 24 and 26, and a second back wall 25 depending perpendicularly from the lid top 31. The second back wall 25 may include a locking tab 19. The second front wall 27, second set of opposed walls 24 and 26, and second back wall 25 are joined at their respective ends and have portions defining a second compartment. The portion 34 having portions defining a second compartment includes a second polyvinyl alcohol foam pad 21 within said compartment and adhesively attached to the top lid 31. The aforementioned adhesive securing the second polyvinyl alcohol foam pad 21 to the top wall is polyvinyl alcohol based. Further the polyvinyl alcohol based adhesive is water soluble for the purpose of dissolving upon insertion into the fixative solution, thus allowing the sandwich to be removed from the cassette 8 upon processing. The top lid 31 may include a plurality of perforations 30. The lid portion 34 forms a second lip 20 for releasably mating with the base portion 33. The lid portion 34 includes a lifting tab 23 and may include a locking tab 22.

The tissue cassette 8 in accordance with the present invention may be of any size or shape with the preferred embodiment being sufficiently rectangular. The tissue cassette 8 may be transparent or of any color and may be constructed, formed, machined, extruded, molded, cast, or otherwise made from any suitable material including but not limited to metal, plastic, fiberglass, composite, or the like.

The engaging member of the transverse slots 14 and 17 of the tissue cassette 8 may be constructed, formed, machined, extruded, molded, cast, or otherwise to receive the locking tabs 22 and 19 respectively as shown in FIGS. 1-3 and may be molded or otherwise constructed in its original forming process as part of the tissue cassette.

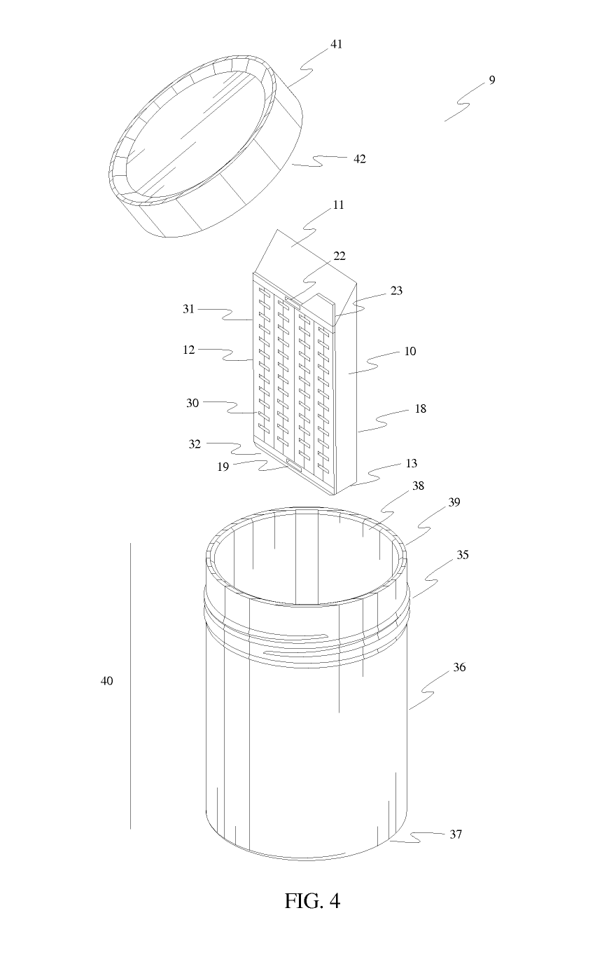

A second embodiment, in accordance with the present invention as illustrated in FIG. 4 includes a cassette storage container 9 with a container bases portion 40 and a container lid portion 41. The container base portion 40 includes a cylindrical wall 36 depending perpendicularly from the bottom wall 37. The container lid portion 41 is made to engage the container base portion 40 by releasably coupling complementary threads 42 and 35 located respectively on the interior of the container lid portion 41 (not shown) and the exterior of the cylindrical wall 36 of the container base portion 40. The top surface of the cylindrical wall 36 forms a rim 39 for the purposes of sealing the container 9 contents during coupling of the container lid portion 41 with the container base portion 40. The container 9 contents may include the tissue cassette 8 and a fixative solution and may reside in the interior portion 38 of the container.

The cassette container 9 in accordance with the present invention may be of any size or shape with the preferred embodiment being sufficiently cylindrical. The storage container 9 may be transparent or any color and may be constructed, formed, machined, extruded, molded, cast, or otherwise made from any suitable material including but not limited to metal, plastic, fiberglass, composite, or the like.

The graph illustrated in FIG. 5 compares the wicking rate of the polyvinyl alcohol foam pads 16 and 21 in cm per a five minute period with that which is currently known in the art. Normalizing the data to 1 shows that the polyvinyl alcohol foam pads 16 and 21 have a 0.9 cm/min wicking rate as compared to 0.05 cm/min, 0.02 cm/min, and 0.05 cm/min for polyurethane (PU), nylon mesh, and lens paper respectively.

The graph illustrated in FIG. 6 compares the absorption rate of the polyvinyl alcohol foam pads 16 and 21 as a percent with that which is currently known in the art. The figure shows that the polyvinyl alcohol pads 16 and 21 have an 1100% absorption rate as compared to 475%, 225%, and 250% by weight for polyurethane, nylon mesh, and lens paper respectively.

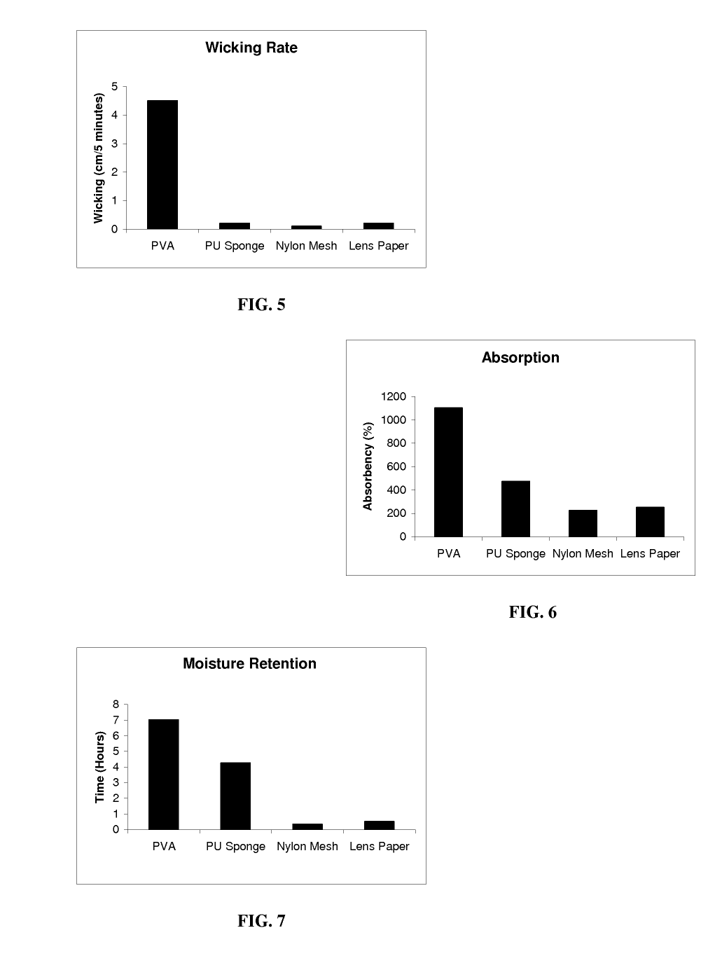

The graph illustrated in FIG. 7 compares the moisture retention rate of the polyvinyl alcohol pads 16 and 21 in hours (hrs) with that which is currently known in the art. The figure shows that the polyvinyl alcohol foam pads 16 and 21 have a moisture retention time of 7 hours as compared to 4.25 hrs, 0.4 hrs, and 0.5 hrs for polyurethane, nylon mesh, and lens paper respectively.

The various embodiments of the present invention as shown in FIGS. 1-7 may be arranged and designed in a wide variety of different configurations that fall within the scope of the present invention, and may be applied to any type of system involving the collection, storage, transportation and/or processing of biopsy tissue samples.

In short the improved biopsy kit provides a unique design for the safe, efficient, and effective handling of biopsy tissue samples. This biopsy kit utilizes a tissue cassette with a removable set of polyvinyl alcohol foam pads for securing and isolating a tissue sample in its original orientation. By utilizing the improved tissue cassette tissue loss is prevented, substantial errors are eliminated and time is saved in processing biopsy tissue samples.

The foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims.

The invention claimed is:

1. An apparatus for the collection, transportation, storage, and processing of a biological specimen, the apparatus comprising:
a tissue cassette having a base member and a cooperable lid member; said base member having an open top perforated bottom wall, opposed side walls, front and back walls, support wall, and slanted front wall; said front wall having a first lid connecting means; said back wall having a second lid connecting means; said bottom wall, said opposed side walls, said front wall depending perpendicularly from said bottom wall and having portions defining a compartment; said bottom wall having an attached polyvinyl alcohol foam pad contained within said portions defining a compartment; said lid member having a perforated top wall, second opposed side walls, second front and back walls; said top wall including a coplanar lifting tab formed at one end thereof; said lid defining a first engaging means depending from and below said one end of said top wall for releasably engaging said first lid connecting means of said back wall; said lid defining a second base engaging means at an opposite end thereof for releasably engaging said second lid connecting means of said base member; said top wall, said second opposed side walls, said second front wall depending perpendicularly from said top wall and having portions defining a second compartment; said top wall having a second attached polyvinyl alcohol foam pad contained within said portions defining a second compartment; said base member and said lid member being attached by a hinge portion along a common side, said base member and said lid member being capable of relative movement about said hinge portion from a first position permitting placement of a specimen in said base member to a second position wherein the open top of said base member is mated with said lid member and engagement means;
a container for holding said tissue cassette; and
a sealing closure lid having means adopted to securely attach said lid to said container.

2. The apparatus of claim 1, wherein said container is adapted to hold one or more of said tissue cassettes.

3. The apparatus of claim 1, wherein said hinge portion is a living hinge.

4. The apparatus of claim 1, wherein said polyvinyl alcohol foam pads are adhesively attached to said bottom portion, and said top portion respectively.

5. The apparatus of claim 4, wherein said adhesive is polyvinyl alcohol based.

6. The apparatus of claim 5, wherein said polyvinyl alcohol based adhesive is water soluble.

7. An apparatus for the collection, transportation, storage, and processing of a biological specimen, the apparatus comprising:
a tissue cassette having a base member and a cooperable lid member; said base member having an open top perforated bottom wall, opposed side walls, front and back walls, support wall, and slanted front wall; said support wall depending perpendicularly from said front wall which in turn depends perpendicularly from said slanted front wall; said front wall having a first lid connecting means; said back wall having a second lid connecting means; said bottom wall, said opposed side walls, said front wall depending perpendicularly from said bottom wall and having portions defining a compartment; said bottom wall having an attached polyvinyl alcohol foam pad contained within said portions defining a compartment; said lid member having a perforated top wall, second opposed side walls, second front and back walls; said top wall including a coplanar lifting tab formed at one end thereof; said lid defining a first engaging means depending from and below said one end of said top wall for releasably engaging said first lid connecting means of said back wall; said lid defining a second base engaging means at an opposite end thereof for releasably engaging said second lid connecting means of said base member; said top wall, said second opposed side walls, said second front wall depending perpendicularly from said top wall and having portions defining a second compartment; said top wall having a second attached polyvinyl alcohol foam pad contained within said portions defining a second compartment; said base member and said lid member being attached by a living hinge portion along a common side, said base member and said lid member being capable of relative movement about said hinge portion from a first position permitting placement of a specimen in said base member to a second position wherein the open top of said base member is mated with said lid member and engagement means;
a container for holding one or more of said tissue cassette; and
a sealing closure lid having means adopted to securely attach said lid to said container thereby sealing the tissue cassette and fixative solution in the interior portion.

8. The apparatus of claim 7, wherein said polyvinyl alcohol foam pads are adhesively attached to said bottom portion, and said top portion respectively.

9. The apparatus of claim 8, wherein said adhesive is polyvinyl alcohol based.

10. The apparatus of claim 9, wherein said polyvinyl alcohol based adhesive is water soluble for the purposes of detaching the polyvinyl alcohol foam pad from the cassette upon insertion into the fixative solution.
